(19) United States
(12) Patent Application Publication
(10) Pub. No.: US 2023/0101402 A1
(43) Pub. Date: Mar. 30, 2023

(54) DEVICE FOR DELIVERING MATERIAL BY APPLYING OPTIMUM INJECT CYCLE

(71) Applicant: ADDOBIO, Seoul (KR)

(72) Inventor: Changok KIM, Gunpo (KR)

(21) Appl. No.: 17/792,127

(22) PCT Filed: Mar. 4, 2020
(86) PCT No.: PCT/KR2020/003057

§ 371 Date: Jul. 11, 2022

(51) Int. Cl. A61M 5/30; A61M 5/20; A61M 39/24

(52) U.S. Cl. CPC A61M 5/30; A61M 5/2053; A61M 39/24; A61M 2037/0007

(57) ABSTRACT

The present invention relates to a device for injecting a material, the device comprising the laser generation unit generates laser beams at a repetition rate of 15 to 25 Hz such that an inject material is injected so as to penetrate into skin through the surface of skin. As described above, a device for delivering a material by applying an optimum inject cycle according to the present invention is advantageous in that a material can be injected at an optimum inject cycle and made to penetrate into skin without a needle, thereby improving the penetration efficiency and minimizing the residual material which has failed to penetrate.

### TECHNICAL FIELD

The disclosure relates to a device for delivering a material based on an optimum inject cycle, and more particularly to a material injecting device that injects a material to efficiently penetrate into skin.

### BACKGROUND ART

As a classic method of delivering a material into tissue through skin of a human, the material has been delivered through a needle penetrating into the skin or a medicine has been applied to the skin.

Recently, as disclosed in Korean Patent No. 1165768, a needleless syringe has been developed and used to inject a material into skin without using a needle.

However, such a conventional needleless syringe has problems in that its configuration is complicated and an efficiency of delivering a medicine is low.

### DISCLOSURE

### Technical Problem

To solve the foregoing problems of a conventional device for delivering a material, an aspect of the disclosure is to provide a device for delivering a material, which has a simple configuration and maximizes an efficiency of material penetration into skin.

### Technical Solution

According to an embodiment of the disclosure, there may be provided a material inject device including: a main body including a power supply; a handpiece including a laser beam generator configured to generate a laser beam with power received from the power supply; and a material inject tip detachably mounted to the handpiece, and configured to inject an inject material accommodated therein at an end thereof as internal pressure is increased by the laser beam emitted from the laser beam generator, wherein the laser beam generator generates the laser beam at a repetition rate of 15 to 25 Hz so that the inject material is injected to penetrate into skin through a skin surface.

Meanwhile, the laser beam generator may be configured to generate the laser beam having a wavelength of 532 nm, 1064 nm, 2900 nm or 2940 nm.

Meanwhile, the inject material may be in a liquid state.

Meanwhile, the material inject tip may include an inject nozzle formed with an injecting hole having an inner diameter of 50 to 500 μm.

Meanwhile, the material inject tip may include: a pressure chamber configured to accommodate liquid therein; a window provided at a first side of the pressure chamber, and configured to allow a laser beam emitted from an outside to pass therethrough and reach the liquid accommodated in the pressure chamber; and a membrane unit provided at the first side of the pressure chamber, and configured to seal the pressure chamber and be transformed by pressure generated as the liquid is irradiated with the laser beam.

Further, the material inject tip may include an inject material accommodating chamber configured to accommodate an inject material, configured to accommodate tattoo dye, and configured for fluid communication with the membrane unit.

In addition, the material inject tip may further include an inject material supply unit detachably mounted to one side of the inject material accommodating chamber to supply the inject material to the inject material accommodating chamber, and the inject material accommodating chamber includes a channel for fluid communication with the inject material supply unit at one side thereof.

Meanwhile, the inject material accommodating chamber may further include a first valve configured to set whether to allow the inject material to flow from the inject-material supply unit toward the inject material accommodating space.

Further, the first valve may include an opening/closing portion configured to seal an opening of the channel at a side of the inject material accommodating chamber, and the opening/closing portion may include an elastic material to be transformed by difference between the internal pressure of the inject material accommodating chamber and the internal pressure of the channel.

Meanwhile, the inject material accommodating chamber may be internally formed with a cylindrical space, the first valve may have a hollow shape and include an outer circumferential surface to come into close contact with an inner surface of the cylindrical space of the inject material accommodating chamber, and the opening/closing portion may be configured to be transformed toward a central axis of the hollow.

Meanwhile, a second valve may be further provided inside the inject material accommodating chamber, and configured to set whether to allow the inject material to flow toward the inject nozzle.

Further, the second valve may include a one-way valve.

Further, the inject material accommodating chamber may further include a stopper configured to press the second valve toward the inject nozzle so that the second valve can be locked in the inject material accommodating chamber

Further, the stopper may be internally formed with a hollow to allow the inject material to move from the inside of the inject material accommodating chamber to the inject nozzle.

### Advantageous Effects

According to the disclosure, a device for delivering a material based on an optimum inject cycle can inject a material to penetrate into skin based on the optimum inject cycle without a needle, thereby having effects on improving a penetration efficiency and also minimizing a remaining material that does not penetrate into the skin.

### DESCRIPTION OF DRAWINGS

FIG. 1 is a perspective view of a material inject device mounted with a material inject tip according to the disclosure.

FIG. 2 is a perspective view of a material inject tip.

FIG. 3 is an exploded perspective view of a material inject tip.

FIG. 4 is an exploded perspective view of a pressure chamber.

FIG. 5 is a cross-sectional view of a pressure chamber.

FIG. 6 is an exploded perspective view of an inject material chamber.

FIG. 7 is a cross-sectional view of an inject material chamber.

FIG. 8 is an enlarged exploded perspective view of an inject nozzle.

FIG. 9 is an operating state view when a material inject tip injects an inject material.

FIG. 10 is an operating state view immediately after a material inject tip injects an inject material.

FIG. 11 is a view showing an alternative example of an inject nozzle.

FIG. 12 is an exploded perspective view of a material inject tip according to another embodiment.

FIG. 13 is a cross-sectional view of the embodiment shown in FIG. 12.

FIG. 14 is an using state view of the embodiment shown in FIG. 12.

FIGS. 15A to 15C are using state views when a material is injected at an optimum cycle.

FIG. 16 is a graph showing a ratio of penetration and remaining materials according to inject cycles.

### MODE FOR INVENTION

Hereinafter, a material inject tip according to embodiments of the disclosure will be described in detail with reference to the accompanying drawings. The names of elements used in the following description may be referred to as other names in the art. However, these elements may be considered as equivalent elements in alternative embodiments as long as they have functional similarity and identity. Further, the reference numerals of the elements are provided for the convenience of description. However, the elements indicated by the reference numerals in the drawings are not limited to the scope shown in the drawings. Similarly, even though some elements in the drawings are modified in alternative embodiments, these elements are considered as equivalent elements as long as they have functional similarity and identity. Further, when elements are regarded as elements that should be naturally included at the level of those skilled in the art, descriptions thereof will be omitted.

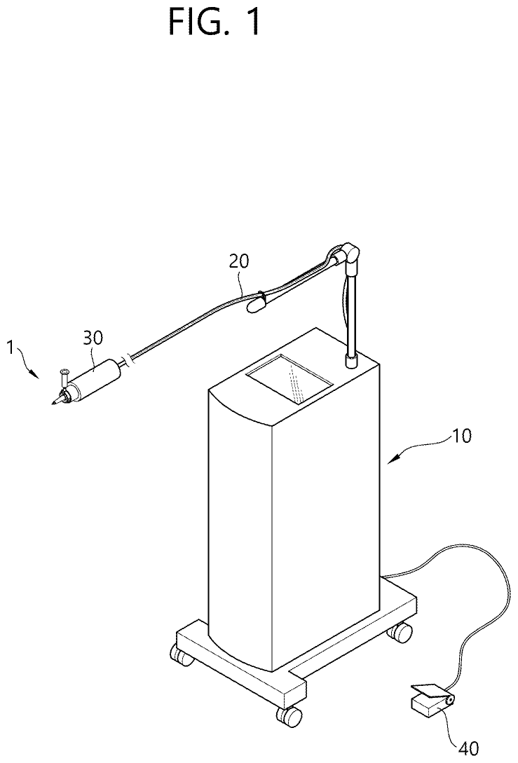

FIG. 1 is a perspective view of a material inject device mounted with a material inject tip 1 according to the disclosure.

As shown therein, a material inject tip 1 according to the disclosure is connected to a laser beam generator, is irradiated with a laser beam from the outside, and internally generates pressure based on the irradiation of the laser beam, thereby injecting a material.

The material inject device may include a main body 10, a foot switch 40, a cable 20, and a handpiece 30.

The main body 10 may include a controller, an interface, and a power supply. The controller may be configured to generate a laser beam in response to a user's input, and configured to control the pattern, cycle, number, etc. of generated pulses of the laser beam according to the inputs. However, the controller has been widely used including a processor, and thus detailed descriptions thereof will be omitted.

The interface may be configured to receive a user's control input, and configured to display information about a currently set state and use. The interface may for example be provided as a display unit with a touchscreen.

The controller may be configured to receive electric power from the outside and supply suitable power to electric components connected to the main body 10 and the handpiece 30.

The foot switch 40 is configured to be stepped on by a user to control the operations of the material inject device. The foot switch 40 is configured to enable a user to control the operations of the material inject device with his/her foot while both hands are used in holding and moving the material inject device wile injecting a material.

The cable 20 is configured to transmit the electric power from the main body 10 to the handpiece 30 (to be described later), and a first side of the cable 20 may be supported on an arm extended from or connected to the main body 10 to prevent interference during a procedure.

The handpiece 30 may include a laser generator (not shown) and a lens (not shown). The laser generator is configured to receive electric power from the outside and generate a laser beam having a certain wavelength. The laser generator may emit a laser beam to fluid accommodated in a pressure chamber 100 (to be described later), so that the internal pressure of the pressure chamber 100 can be instantaneously increased. The wavelength of the laser beam emitted from the laser may be selected among various wavelengths of 532 nm, 1064 nm, 2900 nm, etc. For example, the laser generator may be configured to generate a laser beam having a wavelength of 2940 nm, of which the energy efficiency is the highest for water. However, such a wavelength of the laser beam generated by the laser generator is merely an example, and may be variously selected in consideration of the properties of the liquid in the pressure chamber 100.

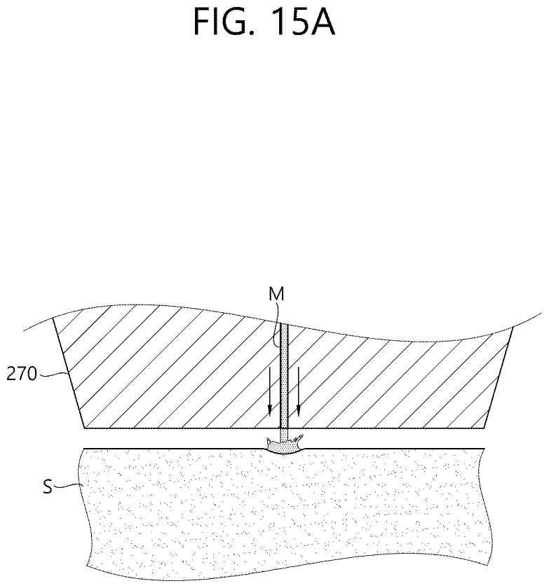

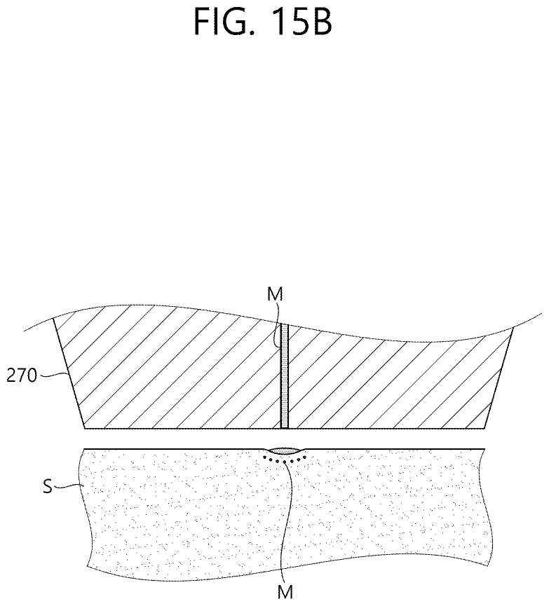

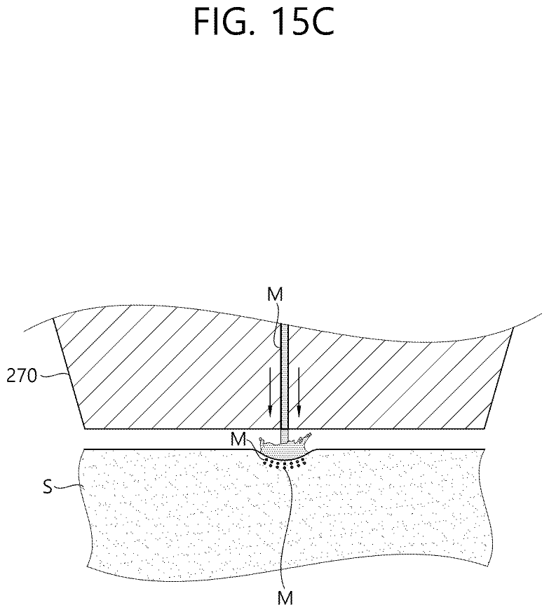

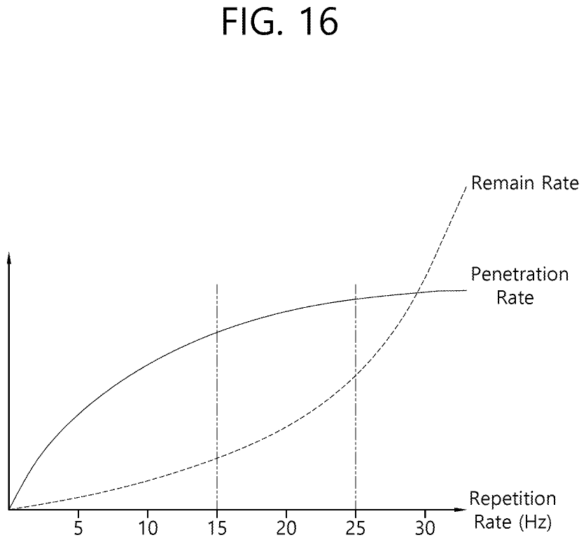

Meanwhile, such operations of the laser beam generator will be described later in detail with reference to FIGS. 15 and 16.

Meanwhile, the lens (not shown) is configured to condense the laser beam emitted from the laser generator, and, for example, the laser beam may be focused on the inner space of the pressure chamber when the material inject tip 1 (to be described later) is mounted to the handpiece 30.

The material inject tip 1 may be mounted at the end of the handpiece 30. The material inject tip 1 may be structured to be easily mounted to and separated from the handpiece 30, and may be replaced with another tip according to users or according to the kinds of materials. In other words, the material inject tip may be selected as requested by a user and mounted at the end of the handpiece 30. The material inject tip 1 may be irradiated with the foregoing laser beam so as to be internally increased in pressure. The material inject tip 1 may be configured to inject the inject material through a nozzle based on the pressure generated therein, so that the injected material can be injected into a skin of human skin. The nozzle has an injecting hole 272, the inner diameter of which is within a predetermined range to inject the material into the skin when the material is injected at high pressure while minimizing the pain of a person who undergoes the procedure.

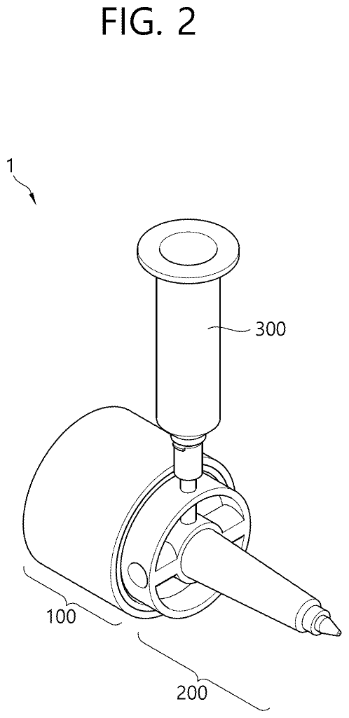

Meanwhile, the elements of the material inject tip 1 will be described below in detail with reference to FIGS. 2 to 11.

FIG. 2 is a perspective view of the material inject tip 1.

Referring to FIG. 2, the material inject tip 1 according to an embodiment of the disclosure is configured to inject a material temporarily accommodated therein by the pressure generated when irradiated with the laser beam.

The material inject tip 1 according to the disclosure may include the pressure chamber 100, an inject material chamber 200, an inject material supply unit 300, and a nozzle cap 400.

The size of the material inject tip 1 may be suitable for a user to precisely position and use the tip 1 during the tattooing procedure. For example, the tip 1 may have an outer appearance shaped like a cylinder, the diameter of which ranges from 1 to 5 cm to be easily gripped by a user.

Each of the pressure chamber 100 and the inject material chamber 200 may approximately be rotationally symmetric, and the pressure chamber 100 and the inject material chamber 200 may be connected to each other in a direction of a rotationally symmetric axis.

The inject material supply unit 300 is configured to store an inject material therein and continuously supply the inject material to the inject material chamber 200 as the inject material accommodated in the inject material chamber 200 is used. The inject material supply unit 300 is configured to naturally move the inject material to the inject material chamber 200 based on difference between the internal pressure of the inject material chamber 200 and the internal pressure of the inject material supply unit 300. The inject material supply unit 300 may for example have a cylindrical shape, and may include a slider valve 310 that seals the inside of the cylinder to continuously supply the inject material while maintaining the internal pressure equal to the atmospheric pressure. The slider valve 310 may naturally move toward a valve of a connection port 220 as the inject material in the cylinder moves outwards, i.e., toward the inject material chamber 200. Therefore, the inject material supply unit 300 is prevented from being contaminated from the outside, and stably supplies the inject material.

The nozzle cap 400 is configured to temporarily accommodate the inject material not to leak out when the inject material is injected into and fully filled in the inject material chamber 200. The nozzle cap 400 may include a filter (not shown) placed therein. The filter may be configured to pass gas but filter liquid. When inject material is injected into the inject material chamber 200, the filter first discharges air filled in the inject material accommodating space, and then filters the inject material injected out of an inject nozzle 270 after the inject material accommodating space is fully filled with the inject material. Therefore, it is possible to prevent contamination from the outside when the material inject tip 1 is initially filled with the inject material. Meanwhile, when the inject material leaks from the inject nozzle 270, a user may determine that the inject material is completely injected into the inject material chamber 200. Then, the nozzle cap 400 is removed when the device for injecting a material 1 is used.

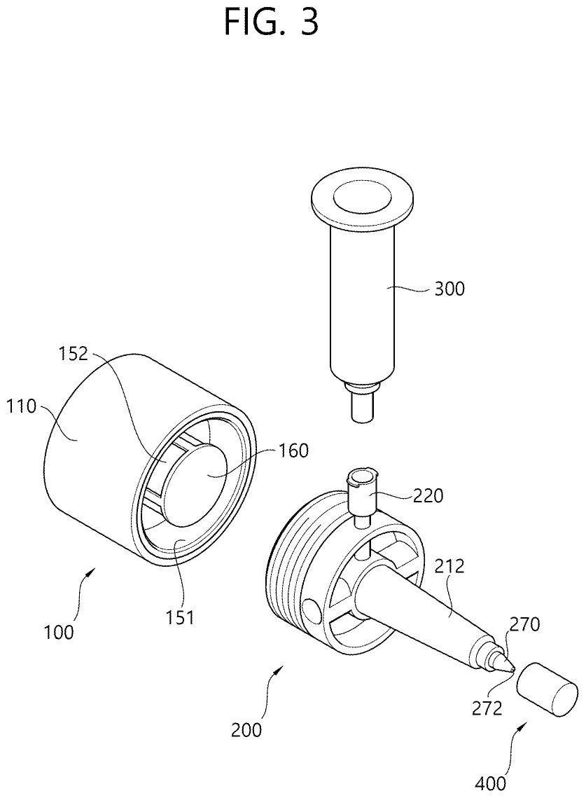

FIG. 3 is an exploded perspective view of the material inject tip 1.

Referring to FIG. 3, the pressure chamber 100 may be connected to the inject material chamber 200, and the pressure chamber 100 and the inject material chamber 200 may be configured to interactively affect their internal pressures when they are connected to each other. The inject material chamber 200 and the pressure chamber 100 may have screw threads or the like publicly known coupling structure to be connected to each other. Further, female/male coupling structures may be respectively provided in the inject material chamber 200 and the pressure chamber 100, thereby facilitating the coupling therebetween. For example, the pressure chamber 100 and the inject material chamber 200 may respectively include a first connector 151 and a second connector 211, which will be described later.

The inject material supply unit 300 may be configured to be connected to a connection port provided at a first side on a lateral surface of the inject material chamber 200 so as to prevent interference when a user grips the material inject tip 1. The inject material supply unit 300 is configured to be easily replaced with a new inject material supply unit 300 when the inject material stored therein is used up.

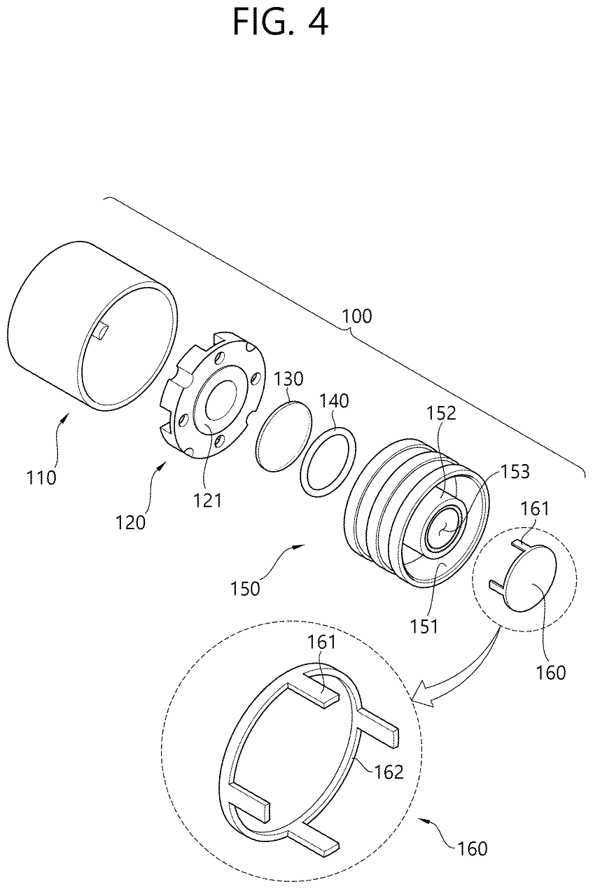

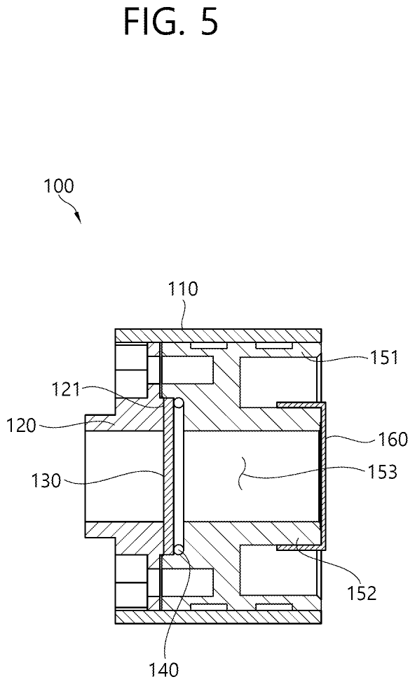

Below, the structure of the pressure chamber will be described in detail with reference to FIGS. 4 and 5.

FIG. 4 is an exploded perspective view of the pressure chamber 100, and FIG. 5 is a cross-sectional view of the pressure chamber 100.

Referring to FIG. 4, the pressure chamber 100 may include a shield 110, an upper cap 120, a window 130, an O-ring 140, a pressure chamber housing 150, and a membrane unit 160.

The shield 110 is configured to generally prevent an impact applied from the outside, and improve a user's grip feeling. The shield 110 may for example be shaped like a hollow configured to surround the lateral side of the pressure chamber housing 150 (to be described later), and may be made of an elastic material.

The upper cap 120 may be coupled to the pressure chamber housing 150 (to be described later), and may be configured to firmly hold the window 130 and the O-ring 140. The upper cap 120 may be generally shaped like a disc, and formed with holes at a center portion to pass the laser beam therethrough. The upper cap 120 may couple with the pressure chamber housing 150 in the direction of the rotationally symmetric axis. The upper cap 120 may have a first side surface formed with a protrusion 121 to press the window 130 and the O-ring 140. On the other hand, the upper cap 120 may have a second side surface provide with a connection structure to which the handpiece 30 is connected from the outside. Meanwhile, such a structure of the upper cap 120 is merely an example, and the upper cap 120 may have various structures for making the window 130 and the O-ring 140 be in close contact with the pressure chamber housing 150.

The window 130 is configured to pass the laser beam coming from the outside. The window 130 may be made of a material appropriately strong enough not to be damaged even when the internal pressure of the pressure chamber 100 is increased by the liquid. The window 130 may for example be made of sapphire glass.

The O-ring 140 is provided between the window 130 and the pressure chamber housing 150 (to be described later) to prevent the liquid accommodated in the pressure chamber housing 150 from leaking out toward the lens. The O-ring 140 may have a widely used configuration, and thus detailed descriptions thereof will be omitted.

The pressure chamber housing 150 may have an inner space 153 to accommodate liquid therein. The pressure chamber housing 150 is provided with the inner space 153, and may include a membrane supporter 152 and the first connector 151.

The inner space 153 is placed in a central portion of the pressure chamber housing 150 so that the laser beam passed through the window 130 can reach the liquid. The pressure chamber housing 150 may generally be rotationally symmetric, and may be provided with the inner space 153 in the central portion thereof. The inner space 153 of the pressure chamber housing 150 is opened facing toward the inject material chamber 200 (to be described later), and its opened portion may be sealed by the membrane unit 160 (to be described later).

The first connector 151 may be provided on an outer side of the pressure chamber housing 150 along a circumferential direction so as to connect with the inject material chamber 200 (to be described later). The inject material chamber 200 (to be described later) may be provided with the second connector 211 to couple with the first connector 151 so that the pressure chamber 100 and the inject material chamber 200 can be connected to each other.

The membrane supporter 152 may be shaped like a hollow forming the inner space 153 and extended a predetermined length in the direction of the rotationally symmetric axis. The membrane supporter 152 may have an end portion facing toward the inject material chamber 200 to support the membrane unit 160 in a thickness direction.

The pressure chamber housing 150 may be made of a material, the strength of which is relatively greater than that of the membrane unit 160, so that change in shape due to the increased internal pressure can be focused on the membrane unit 160 (to be described later) when the internal pressure of the pressure chamber housing 150 is increased.

The membrane unit 160 is configured to seal the inner space 153 of the pressure chamber housing 150, and transfer the pressure to a second side, i.e., to an inject material accommodating space 230 in the inject material chamber 200 as transformed when the inner space 153 is increased in pressure. The membrane unit 160 may include a membrane, a membrane cap, and a membrane guide.

The membrane is shaped like a disc, and has a first side facing the inner space 153 and a second side to be in contact with the inject material accommodating space 230 of the inject material chamber 200 (to be described later).

The membrane cap may be configured to partially surround the membrane supporter 152 of the pressure chamber housing 150. A user fully fills the inner space 153 with liquid, and then covers the membrane supporter 152 with the membrane unit 160. In this case, due to surface tension, the liquid may be a little convexedly filled in the inner space 153 and protrude more than the end of the membrane supporter 152. When the membrane supporter 152 in this state is covered with the membrane unit 160, a membrane cap 162 seals the inner space 153 while preventing gas from flowing into the inner space 153.

A membrane guide 161 may be formed a predetermined length in a direction perpendicular to the membrane. The membrane guide 161 may guide the membrane unit 160 to a sealing position when a user installs the membrane unit 160 to the membrane supporter 152.

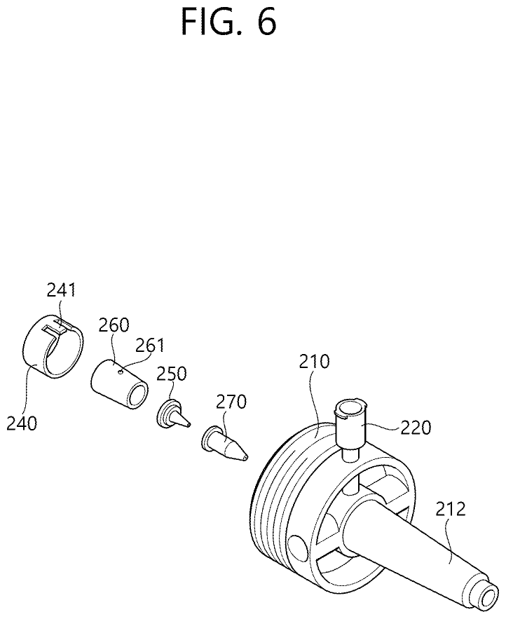

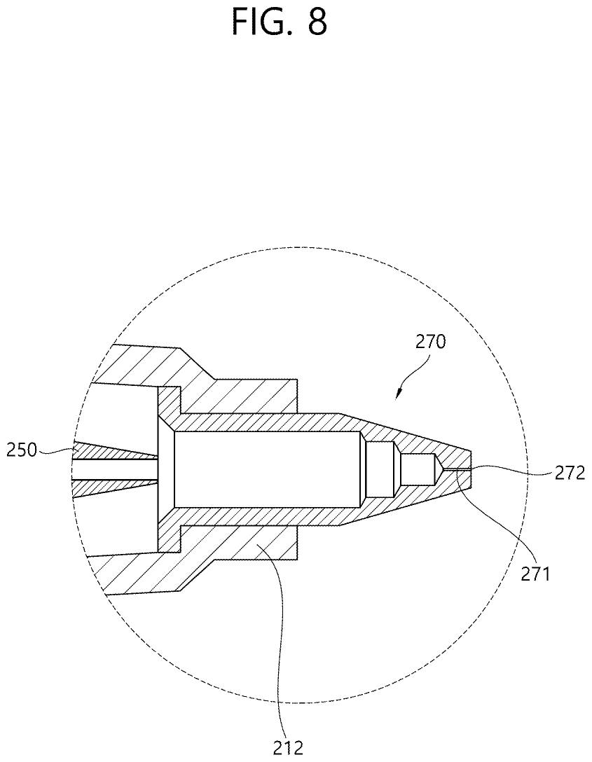

Below, the inject material chamber 200 will be described in detail with reference to FIGS. 6 and 8.

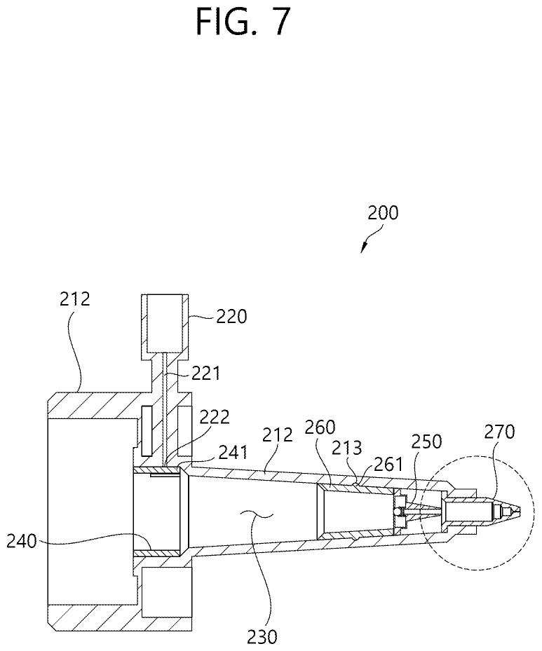

FIG. 6 is an exploded perspective view of the inject material chamber 200, FIG. 7 is a cross-sectional view of the inject material chamber 200, and FIG. 8 is an enlarged exploded perspective view of the inject nozzle 270.

As shown therein, the inject material chamber 200 may include an inject material chamber housing 210, the inject material accommodating space 230, the connection port 220, a channel 222, a first valve 240, a second valve 250, a stopper 260, and the inject nozzle 270.

The inject material chamber housing 210 is structured to accommodate inject material in the inject material chamber 200, and used as a base on which the other elements of the inject material chamber 200 are provided.

The inject material chamber housing 210 is generally rotationally symmetric, and has a first side, to which the foregoing pressure chamber 100 is connected, and a second side, in which the inject nozzle 270 is provided to inject an inject material as the internal pressure increases, along a rotation central axis. The inject material chamber housing 210 may include an extended portion 212 extended in the direction of the rotation central axis so as to have the inject nozzle 270. The extended portion 212 may be shaped like a hollow corn, and allow the inject material to move therein.

The inject material accommodating space 230 may be configured to accommodate the inject material therein, and may be a part of the inner space of the inject material chamber housing 210.

The connection port 220 may be configured to connect with the foregoing inject material supply unit 300, and may be provided on the first side surface of the inject material chamber housing 210. The connection portion 220 may be configured to easily couple with and separate from the inject material supply unit 300.

The channel 222 may be formed penetrating the inject material chamber housing 210 in a radial direction of the rotational symmetry so that a liquid inject material can flow inside the connection port 220 and the inject material accommodating space 230. Therefore, fluid communication is achieved between the inject material supply unit 300 and the inject material accommodating space 230 through the channel 222. The d has a first side formed from an opening 222 at the lateral side of the inject material accommodating space 230, and a second side connected up to the opening 222 formed at an end portion of the connection port 220.

The first valve 240 may be configured to set the direction of the inject material flowing from the inject material supply unit 300 into the inject material accommodating space 230. The first valve 240 may for example be configured to make the inject material flow from the inject material supply unit 300 toward the inject material accommodating space 230. The first valve 240 may for example be shaped like a hollow and fitted to the inner wall of the inject material accommodating space 230. The first valve 240 may include an opening/closing portion 241 at a first side to open and close the opening 222 of the channel 222 formed in the inner wall of the inject material accommodating space 230. The opening/closing portion 241 may be cut in the form of a pair of notches on the hollow-shaped first side and may be bent by external force.

When the first valve 240 is provided in the inject material accommodating space 230, the opening 222 of the channel 222 may be closed by the opening/closing portion 241. Meanwhile, when force based on the internal pressure of the channel 222 is greater than resistance based on the internal pressure of the inject material accommodating space 230 and resilience based on the elasticity of the opening/closing portion 241, the opening/closing portion 241 may be bent toward the inject material accommodating space 230 so that the inject material can flow from the channel 222 toward the inject material accommodating space 230.

However, such a configuration of the first valve 240 is merely an example, and the first valve 240 may alternatively be provided as a one-way valve or a check valve to make the inject material flow from the inject material supply unit 300 to the inject material accommodating space 230.

The second valve 250 may be configured to set the direction of the inject material flowing from the inject material accommodating space 230 toward the inject nozzle 270. The second valve 250 may be provided as inserted in a space formed inside the extended portion 212 of the foregoing inject material chamber housing 210. The second valve 250 may be configured as a one-way valve by which the inject material is allowed to flow toward the inject nozzle 270 in the direction of the rotationally symmetric axis but prevented from flowing in the reverse direction. The second valve 250 may for example be provided as a check valve including a ball and a spring. However, the check valve is merely an example, and the second valve 250 may alternatively be provided as various configurations for setting the flowing direction.

The stopper 260 may be configured to stably lock the foregoing second valve 250 inside the extended portion 212. The stopper 260 may have a tapering shape, in which the diameter of a first side end portion is gradually decreased, corresponding to the inner space of the extended portion 212. The stopper 260 may include a stopper locking portion 261 protruding from one side of the outer surface thereof and firmly locked to the inner surface of the extended portion 212, and the extended portion 212 may include a stopper locking groove 213 formed on the inner surface thereof and corresponding to the stopper locking portion 261.

The inject nozzle 270 is configured to inject the inject material outwards. One side of the inject nozzle 270 may be connected to the extended portion 212 of the foregoing inject material chamber housing 210. The end portion of the inject nozzle 270 may be cut to have a plane surface not to be inserted into skin even though it comes into contact with the skin. The inject nozzle 270 may include an inject channel 271 formed at a central side thereof, and an inject hole formed at the end portion thereof to inject the inject material M passed through the inject channel 271. The inject hole may be formed to have an inner diameter of, for example, 500 μm to 50 μm, and may be internally subjected to a coating to make the inject material smoothly flow. Meanwhile, the inject nozzle 270 may be configured to inject a uniform amount of inject material even in repeatedly use. For example, the inject nozzle 270 may be made of metal.

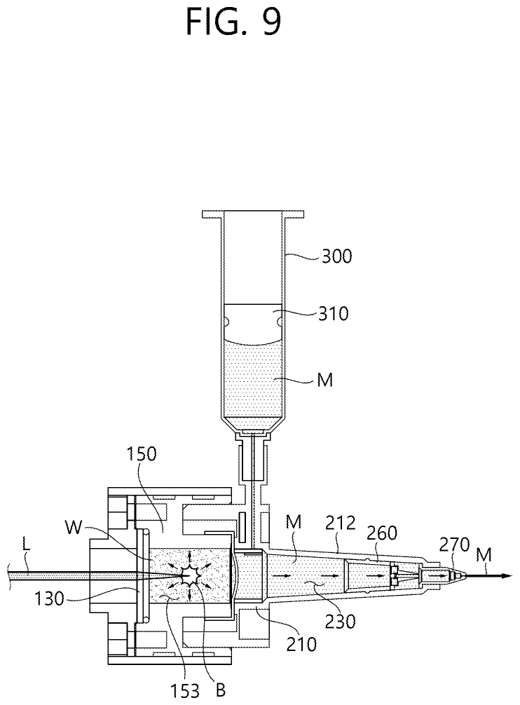

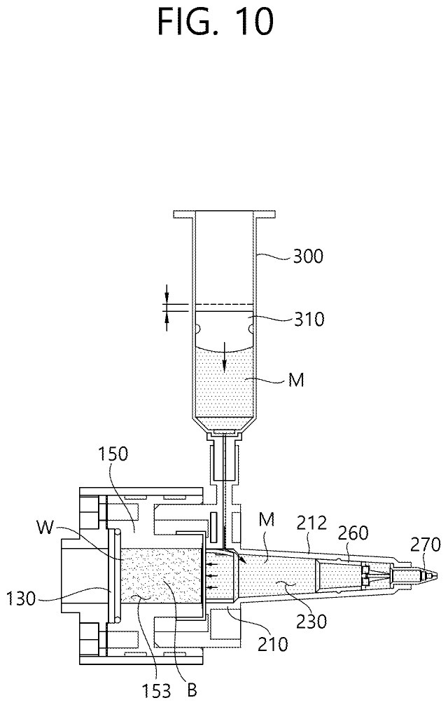

Below, the operations of the material inject tip 1 according to the disclosure will be described in detail with reference to FIGS. 9 and 10.

FIG. 9 is an operating state view when the material inject tip 1 injects an inject material, and FIG. 10 is an operating state view immediately after the material inject tip 1 injects an inject material.

Referring to FIG. 9, the material inject tip 1 may be used with the inject material supply unit 300 connected thereto, and the handpiece 30 may be connected to the first side of the pressure chamber 100. However, the illustration of the handpiece 30 is partially omitted for convenience of description.

First, when the material inject tip 1 is used, the inner space 153 of the pressure chamber 100 is fully filled with liquid, e.g., water W, in which hot water of 20° C. to 80° C. may be used to minimize the generation of bubbles. Meanwhile, the inject material accommodating space 230 is also fully filled with the inject material M to make ready for the operation.

Then, when a user makes an input, for example, steps on the foot switch 40 to apply the pulses of a laser beam L, the laser beam causes bubbles B to be generated inside the pressure chamber 100 and the generation of bubbles instantaneously increase the internal pressure of the pressure chamber 100. Then, the pressure increased inside the pressure chamber 100 causes the membrane unit 160 to be transformed toward the inject material accommodating space 230, thereby transferring the pressure to the inject material accommodating space 230. When the pressure is increased inside the inject material accommodating space 230, the inject material M is injected through only one outlet, i.e., the inject nozzle 270.

Referring to FIG. 10, after the inject material M is injected by one application of the laser beam, the bubbles B generated inside the inner space 153 of the pressure chamber 100 disappear and the pressure is decreased. Further, the membrane unit 160 returns toward the inner space 153, and thus the pressure inside the inject material accommodating space 230 is momentarily decreased. In this case, the pressure inside the inject material accommodating space 230 is momentarily lower than the pressure inside the channel 222, and thus the first valve 240 is opened so that the inject material M can flow from the channel 222 to the inject material accommodating space 230, thereby refilling the inject material accommodating space 230 with the inject material as much as injected out once. In this case, the second valve 250 prevents air and foreign materials from being introduced from the inject nozzle 270 into the inject material accommodating space 230, so that the pressure decreased inside the inject material accommodating space 230 is entirely used in moving the inject material M from the inject material supply unit 300 to the inject material accommodating space 230.

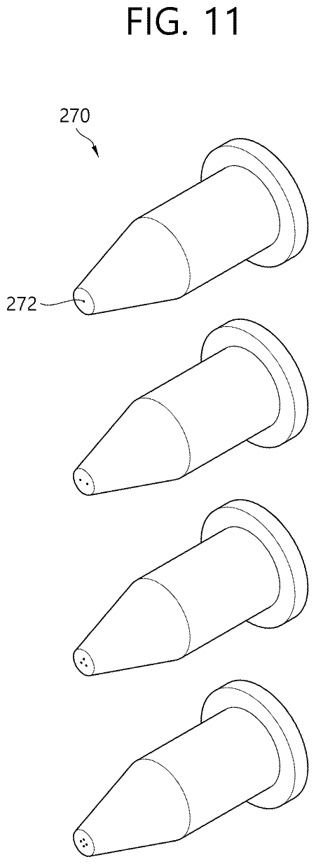

Below, an alternative example of the inject nozzle 270 will be described with reference to FIG. 11.

FIG. 11 is a view showing an alternative example of an inject nozzle.

As shown therein, the number of injecting holes 272 in the inject nozzle 270 and each diameter of injecting holes 272 may be variously set. When the number of injecting holes 272 is increased, the pressure change inside the pressure chamber 100 is taken into account to set the total cross-sectional areas of the channels the injecting holes 272 have and set the arrangement of the injecting holes 272. However, this is merely an example, and the number and diameter of injecting holes 272 may be variously combined.

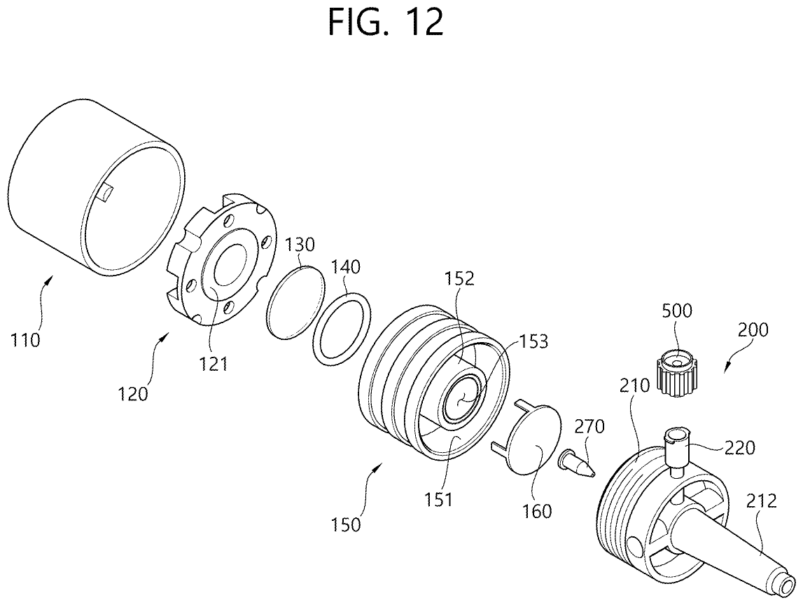

Below, the material inject tip 1 according to another embodiment of the disclosure will be described with reference to FIGS. 12 to 14. Meanwhile, this embodiment may also include the same elements as those described in the foregoing embodiments, and therefore different elements will be described in detail without describing the same elements to avoid repetitive descriptions.

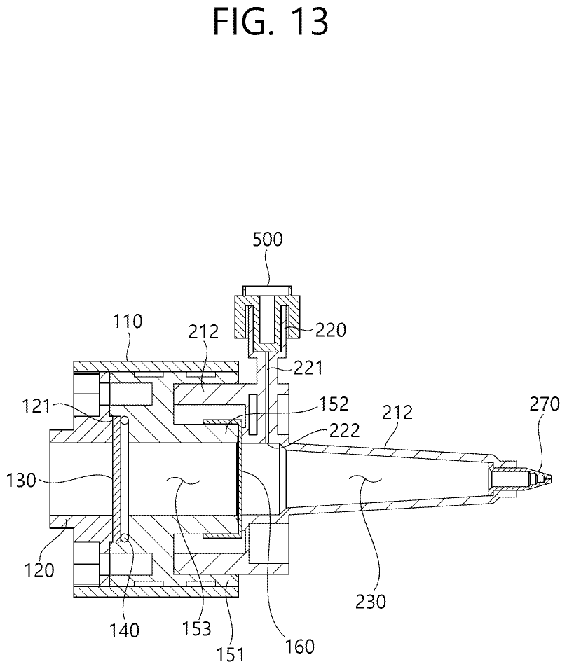

FIG. 12 is an exploded perspective view of a material inject tip according to another embodiment, and FIG. 13 is a cross-sectional view of the embodiment shown in FIG. 12.

Unlike the foregoing embodiments, the material inject tip 1 according to this embodiment is configured to be directly used by a user in the state that the inject material chamber 200 is filled with the inject material. In this embodiment, the inject material chamber 200 may include the inject material chamber housing 210, the connection port 220, a connection port cap 500, and the inject nozzle 270.

The inject material chamber housing 210 may be configured similarly to that of the foregoing embodiment, and may include a space to accommodate inject material therein.

In this embodiment, the connection port 220 may couple with the connection port cap 500. First, a user may inject the inject material M through the connection port 220. When the inject material is filled in the inject material accommodating space through the connection port 220, a user stops injecting the inject material and seals up the connection port 220 with the connection port cap 500.

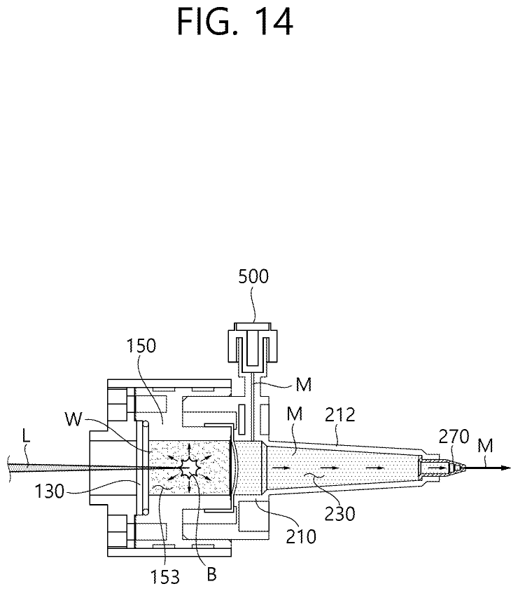

FIG. 14 is an using state view of the embodiment shown in FIG. 12. As shown therein, the inject material M may be accommodated in the connection port 220, i.e., in the channel 221 and the inject material accommodating space 230. The connection port 220 may be sealed up with the connection port cap 500. Like the foregoing embodiments, when the pressure chamber 100 is irradiated with the laser beam L and increased in pressure, the pressure may be transferred to the inject material accommodating space 230 by the membrane unit 160. As the pressure of the inject material accommodating space 230 is increased, the inject material may be finally discharged through only one outlet, i.e., the inject nozzle 270.

FIGS. 15A to 15C are using state views when a material is injected at an optimum cycle. In these drawings, some parts may be exaggerated or reduced for the convenience of description.

FIG. 15A shows an operation of a material inject tip when the laser beam L is initially emitted from the laser beam generator. As described above, the material spry tip is internally increased in pressure when irradiated with the laser beam L, and therefore the inject material M is injected through the inject nozzle 270.

While the inject material M injected out through the inject nozzle 270 collides with the surface of skin, some injected material M penetrates into tissue through the surface of skin, but the rest remains outside the skin. In this case, the surface of skin may momentarily become a state of being recessed by the injected inject material M.

FIG. 15B shows a state of a pulse cycle in which the laser beam L is not emitted from the laser beam generator. As shown therein, the surface of skin is kept recessed for a certain period of time after the pulses of the laser beam L is applied to the material spry tip. In this case, the amount of material additionally penetrating into the skin tissue S through the surface of skin is insignificant.

FIG. 15C shows that the material inject tip is not changed in position after the state shown in FIG. 15B, and injects the inject material M as being irradiated with the laser beam L again. When a predetermined period of time elapses from the state shown in FIG. 15B without the irradiation of the laser beam L, the skin returns to its original shape by the elasticity of the skin. On the other hand, if the material is injected again as shown in FIG. 15C in the state shown in FIG. 15B, i.e., before the surface of skin returns to its original shape, pressure is applied by the inject material M injected out from the inject nozzle 270 in addition to the inject material M remaining on the surface of the skin, thereby maximizing the amount of inject material M penetrating into the skin.

FIG. 16 is a graph showing a ratio of penetration and remaining materials according to inject cycles.

As described above, the amount of material penetrating into the skin may be varied depending on the inject cycles of the material. Some injected material penetrates into the tissue S of the skin, but the rest remains outside the skin. It is difficult to reuse the remaining inject material M because of external contamination. Therefore, the irradiation cycle of the laser beam L may be set to maximize the amount of material penetrating into the tissue S of the skin and minimize the amount of material remaining outside the skin.

Referring back to FIG. 16, the repetition rate of the laser beam L tends to coincide with a repetition rate at which the inject nozzle 270 injects the inject material M, and it is therefore possible to adjust the repetition rate of the inject material M by controlling the laser beam L.

When the number of inject times per second is increased up to 25 Hz, i.e., 25 times per second based on the repetition rate of the laser beam L, a penetration rate of the inject material M tends to increase. However, the increasing tendency of the penetration rate of the inject material M decreases as the number of inject times per second is further increased. On the other hand, the remaining amount of inject material M Remain Rate tends to increase as the number of inject times per second of the laser beam L is increased, and tends to suddenly increase at 25 Hz or higher. This is because a total amount of inject material M increases but the penetration rate of the inject material M limitedly increases as the repetition rate of the laser beam L becomes higher. Therefore, a user may set the repetition rate of the laser beam L within a range of 15 to 25 Hz so that the inject cycle of the inject material M can be selected within a range of 15 to 25 times per second.

Meanwhile, the foregoing inject material M may include various liquid materials, for example, medicine delivered through skin, tattoo dye, etc., which can be injected out from the inject nozzle 270 as the pressure increases,

As described above, a device for delivering a material based on an optimum inject cycle according to the disclosure can inject a material to penetrate into skin based on the optimum inject cycle without a needle, thereby having effects on improving a penetration efficiency and also minimizing a remaining material that does not penetrate into the skin.

1. A material inject device comprising:
a main body comprising a power supply;
a handpiece comprising a laser beam generator configured to generate a laser beam with power received from the power supply; and
a material inject tip detachably mounted to the handpiece, and configured to inject an inject material accommodated therein at an end thereof as internal pressure is increased by the laser beam emitted from the laser beam generator,
wherein the laser beam generator generates the laser beam at a repetition rate of 15 to 25 Hz so that the inject material is injected to penetrate into skin through a skin surface.

2. The material inject device of claim 1, wherein the laser beam generator is configured to generate the laser beam having a wavelength of 532 nm, 1064 nm, 2900 nm or 2940 nm.

3. The material inject device of claim 2, wherein the inject material is in a liquid state.

4. The material inject device of claim 3, wherein the material inject tip comprises an inject nozzle formed with an injecting hole having an inner diameter of 50 to 500 μm.

5. The material inject device of claim 4, wherein the material inject tip comprises:
a pressure chamber configured to accommodate the liquid therein;
a window provided at one side of the pressure chamber, and configured to allow the laser beam emitted from an outside to pass therethrough and reach the liquid accommodated in the pressure chamber; and
a membrane unit provided one side of the pressure chamber, and configured to seal the pressure chamber and be transformed by pressure generated as the liquid is irradiated with the laser beam.

6. The material inject device of claim 5, wherein the material inject tip may comprise an inject material accommodating chamber configured to accommodate an inject material, configured to accommodate tattoo dye, and configured for fluid communication with the membrane unit.

7. The material inject device of claim 6, wherein
the material inject tip further comprises an inject material supply unit detachably mounted to one side of the inject material accommodating chamber to supply the inject material to the inject material accommodating chamber, and
the inject material accommodating chamber comprises a channel for fluid communication with the inject material supply unit at one side thereof.

8. The material inject device of claim 7, wherein the inject material accommodating chamber further comprises a first valve configured to set whether to allow the inject material to flow from the inject material supply unit toward the inject material accommodating space.

9. The material inject device of claim 8, wherein
the first valve comprises an opening/closing portion configured to seal an opening of the channel at a side of the inject material accommodating chamber, and
the opening/closing portion comprises an elastic material to be transformed by difference between the internal pressure of the inject material accommodating chamber and the internal pressure of the channel.

10. The material inject device of claim 9, wherein
the inject material accommodating chamber is internally formed with a cylindrical space,
the first valve has a hollow shape and comprise an outer circumferential surface to come into close contact with an inner surface of the cylindrical space of the inject material accommodating chamber, and
the opening/closing portion is configured to be transformed toward a central axis of the hollow.

11. The material inject device of claim 9, further comprising a second valve provided inside the inject material accommodating chamber, and configured to set whether to allow the inject material to flow toward the inject nozzle.

12. The material inject device of claim 11, wherein the second valve comprises a one-way valve.

13. The material inject device of claim 12, wherein the inject material accommodating chamber further comprises a stopper configured to press the second valve toward the inject nozzle so that the second valve can be locked in the inject material accommodating chamber.

14. The material inject device of claim 13, wherein the stopper is internally formed with a hollow to allow the inject material to move from an inside of the inject material accommodating chamber to the inject nozzle.
